(19) United States
(12) Patent Application Publication
(10) Pub. No.: US 2025/0009947 A1
(43) Pub. Date: Jan. 9, 2025

(54) PERITONEAL DIALYSIS SYSTEM INCLUDING A PATIENT LINE FILTER HAVING A MEMBRANE SHEET

(71) Applicants: BAXTER INTERNATIONAL INC., Deerfield, IL (US); BAXTER HEALTHCARE SA, Glattpark (Opfikon) (CH)

(72) Inventors: Steffen WAGNER, Deerfield, IL (US); Ralf FLIEG, Deerfield, IL (US); Reinhold BUCK, Deerfield, IL (US); Christof BECK, Deerfield, IL (US); Rainer BLICKLE, Deerfield, IL (US); Bernd KRAUSE, Deerfield, IL (US); Torsten KNOER, Deerfield, IL (US)

(21) Appl. No.: 18/710,721

(22) PCT Filed: Nov. 18, 2022
(86) PCT No.: PCT/US22/80130

§ 371 Date: May 16, 2024

Related U.S. Application Data
(60) Provisional application No. 63/291,018, filed on Dec. 17, 2021.

(51) Int. Cl. A61M 1/16; A61M 1/28

(52) U.S. Cl. CPC A61M 1/1631; A61M 1/28; A61M 2205/7518

(57) ABSTRACT

A peritoneal dialysis (“PD”) system (10) includes a PD machine (20); a patient line (50) extending from the PD machine (20); and a filter set (100) including a filter housing (102) having an upper housing plate (102u) and a lower housing plate (102l), and a filter membrane (112, such as a sterilizing grade or a bacteria reduction filter membrane) located between the upper housing plate (102u) and the lower housing plate (102l), the filter set (100) further including a lumen-side connector (104) configured to connect to the patient line (50), the lumen-side connector (104) connected to the filter housing (102) via at least one of a fresh PD fluid tube (106a) or a used PD fluid tube (106b). A method for manufacturing the filter set (100) is also disclosed.

### PRIORITY CLAIM

The present application claims priority to and the benefit of U.S. Provisional Application No. 63/291,018, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated. e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online or PD machines or cyclers that employ disinfection may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The fresh and used PD fluid lumens may again be reusable or disposable. In the instance in which the fresh and used PD fluid lumens are reusable, the lumens terminate with a patient line connector that connects to a lumen-side connector of the filter set. The lumen-side connector in one embodiment includes a fresh PD fluid port for communication with the fresh PD fluid lumen of the dual lumen patient line and a used PD fluid port for communicating with the used PD fluid lumen of the dual lumen patient line. The lumen-side connector also includes threads for threadingly engaging mating threads of patient line connector. The threading of the patient line connector to the lumen-side connector seals mating ports of the patient line connector to the fresh and used PD fluid ports of the lumen-side connector in one embodiment, e.g., via one or more gasket.

A pair of tubes, including a fresh PD fluid tube and a used PD fluid tube, extend from the lumen-side connector to a filter housing. The fresh and used PD fluid tubes are rigid in one embodiment and are bent so as to position the lumen-side connector and filter housing relative to each other in a desired manner. The filter housing is in one embodiment a thin structure having an upper housing plate and a lower housing plate. Each plate has a slightly raised compartment for receiving PD fluid. The raised compartments are surrounded by the perimeters of the upper and lower plates in one embodiment. The plates may also be molded to each have a cylindrical tube receiver. The cylindrical tube receivers may be angled to match a bent angle of the fresh or used PD fluid tube that is received. The plates may be molded plastic and a sealed together along their mating perimeters. The perimeter of one of the upper plate or the lower plate may be formed with a polygonal, e.g., rectangular, tongue that fits into a like-shaped groove formed in the other of upper plate or the lower plate. The tongue and groove fitting helps to center the upper and lower plates together during the sealing process. The upper and lower plates may be ultrasonically sealed, heat sealed or solvent bonded together.

A filter membrane is sealed along its peripheral edge between the upper and lower plates. The filter membrane is a flat sheet in one embodiment that roughly bisects the upper and lower plates. The filter membrane may be a sterilizing grade or a bacteria reduction hydrophilic membrane, which may be formed with porous walls having a pore size of about 0.2 micron through which the fresh PD fluid flows for further filtration.

The lower plate may be molded with a second cylindrical tube receiver for receiving a transfer set-side tube that extends to a transfer set-side connector. The transfer set-side tube, like the fresh and used PD fluid tubes, may be rigid and bent so that the transfer set-side connector and the filter housing are angled relative to each other in a desired manner. The second cylindrical tube receiver may likewise be angled to match an angle formed by the transfer set-side tube. The transfer set-side connector in turn either connects directly to a mating connector of the patient's transfer set or to a mating connector of a short tube placed between the filter housing and the patient's transfer set. The transfer set-side connector may alternatively simply be a port to which the short tube extends over for welding to the port.

During a patient fill, fresh PD fluid flows from the fresh PD fluid lumen, through the lumen-side connector, through the fresh PD fluid tube and into the raised compartment of the upper plate. Inside the raised compartment, the fresh PD fluid is forced under positive pressure from the PD fluid pump through the filter membrane sheet and into the raised compartment of the lower plate. The filter membrane sheet is sized to provide sufficient filtration over multiple patient fills while being small enough not to present discomfort to the patient who is likely sleeping during treatment.

The final filtered fresh PD fluid flows from raised compartment of the lower plate out the transfer set-side tube and the transfer set-side connector into the patient's transfer set, either directly or via the short, flexible tube. The hydrophilic nature of the filter membrane prevents air from migrating across the membrane once the membrane is fully wetted with fresh PD fluid and thus serves a secondary final stage air removal purpose. If needed however, it is contemplated to provide one or more hydrophobic membrane upstream of the filter membrane (from a fresh PD fluid standpoint), e.g., along the surface of the top plate. The one or more hydrophobic membrane allows air to be vented to atmosphere prior to the fresh PD fluid flowing through the filter membrane.

Used PD fluid removed through the patient's transfer set enters the raised compartment of the lower plate of the filter set via the transfer set-side connector and transfer set-side tube and flows under negative pressure via the PD fluid pump from the lower cavity, through the used PD fluid tube, through the used PD fluid port of the lumen-side connector and the used PD fluid lumen, back to the PD machine or cycler. The PD machine or cycler pumps the used PD fluid under positive pressure to drain. The used PD fluid does contact the underside of the filter membrane sheet but does so in a tangential manner, wherein fibrin, proteins and other particulates within the patient's effluent do not tend to be trapped by or caught on the filter membrane. The filter membrane accordingly remains viable over the course of multiple fills of a treatment prior to being discarded with the filter set.

In light of the disclosure set forth herein, and without limiting the disclosure in any way, in a first aspect of the present disclosure, which may be combined with any other aspect described herein, or portion thereof, a peritoneal dialysis (“PD”) system includes a PD machine: a patient line extending from the PD machine; and a filter set including a filter housing having an upper housing plate and a lower housing plate, and a filter membrane located between the upper housing plate and the lower housing plate, the filter set further including a lumen-side connector configured to connect to the patient line, the lumen-side connector connected to the filter housing via at least one of a fresh PD fluid tube or a used PD fluid tube.

In a second aspect of the present disclosure, which may be combined with any other aspect described herein, or portion thereof, the patient line is a dual lumen patient line including a fresh PD fluid lumen placed in fluid communication with the fresh PD fluid tube of the filter set, the dual lumen patient line further including a used PD fluid lumen placed in fluid communication with the used PD fluid tube of the filter set.

In a third aspect of the present disclosure, which may be combined with any other aspect described herein, or portion thereof, the fresh PD fluid lumen is placed in fluid communication with the fresh PD fluid tube of the filter set via a fresh PD fluid port of the lumen-side connector, and wherein the used PD fluid lumen is placed in fluid communication with the used PD fluid tube of the filter set via a used PD fluid port of the lumen-side connector.

In a fourth aspect of the present disclosure, which may be combined with any other aspect described herein, or portion thereof, the fresh PD fluid port and the used PD fluid port are surrounded by a shroud of the lumen-side connector, the shroud including threads for mating with a patient line connector.

In a fifth aspect of the present disclosure, which may be combined with any other aspect described herein, or portion thereof, the fresh PD fluid port and the used PD fluid port are surrounded by a shroud of the lumen-side connector, the shroud including a keyed opening for receiving a patient line connector in a desired orientation.

In a sixth aspect of the present disclosure, which may be combined with any other aspect described herein, or portion thereof, the PD system includes a compressible gasket configured to seal around the fresh and used PD fluid ports between the lumen-side connector and a patient line connector.

In a seventh aspect of the present disclosure, which may be combined with any other aspect described herein, or portion thereof, at least one of the fresh PD fluid tube or the used PD fluid tube is rigid.

In an eighth aspect of the present disclosure, which may be combined with any other aspect described herein, or portion thereof, at least one of the fresh PD fluid tube or the used PD fluid tube is bent so that the lumen-side connector and the filter housing are positioned relative to each other in a desired manner.

In a ninth aspect of the present disclosure, which may be combined with any other aspect described herein, or portion thereof, the filter membrane is a sheet-shaped hydrophilic membrane, and wherein the upper housing plate and the lower housing plate include raised compartments for receiving PD fluid.

In a tenth aspect of the present disclosure, which may be combined with any other aspect described herein, or portion thereof, the upper housing plate and the lower housing plate are formed and sealed along with the filter membrane to form the filter housing via a combined online sealing and deep-drawing process.

In an eleventh aspect of the present disclosure, which may be combined with any other aspect described herein, or portion thereof, the filter housing is configured such that used PD fluid flows tangentially along the filter membrane.

In a twelfth aspect of the present disclosure, which may be combined with any other aspect described herein, or portion thereof, the PD system includes at least one hydrophobic membrane positioned to vent air from the fresh PD fluid upstream from the filter membrane.

In a thirteenth aspect of the present disclosure, which may be combined with any other aspect described herein, or portion thereof, the filter set is configured to connect directly to a patient's transfer set, or wherein the filter set includes a flexible tube configured to connect to the patient's transfer set.

In a fourteenth aspect of the present disclosure, which may be combined with any other aspect described herein, or portion thereof, the filter set includes a transfer set-side connector for connecting to a patient's transfer set, the transfer set-side connector connected to the filter housing via a transfer set-side tube.

In a fifteenth aspect of the present disclosure, which may be combined with any other aspect described herein, or portion thereof, the PD machine includes a pressure sensor positioned to sense the pressure of fresh PD fluid downstream from the filter membrane during a patient fill.

In a sixteenth aspect of the present disclosure, which may be combined with any other aspect described herein, or portion thereof, the filter membrane is a sterilizing grade filter membrane or a bacteria reduction filter membrane.

In a seventeenth aspect of the present disclosure, which may be combined with any other aspect described herein, or portion thereof, a filter set for connecting to a patient line includes a filter housing including an upper housing plate and a lower housing plate: a filter membrane in the shape of a sheet located between the upper housing plate and the lower housing plate; and a lumen-side connector configured to connect to the patient line, the lumen-side connector connected to the filter housing via at least one of a fresh PD fluid tube or a used PD fluid tube.

In an eighteenth aspect of the present disclosure, which may be combined with any other aspect described herein, or portion thereof, a method of manufacturing a filter set for connecting to a patient line includes heating at a softening temperature and applying a vacuum to form a plurality of upper housing plates and lower housing plates: extending a filter membrane in the shape of a sheet between each of the formed upper housing plates and the formed lower housing plates; and heating at a sealing temperature and sealing the upper housing plates to the lower housing plates so as to seal in place the filter membranes.

In a nineteenth aspect of the present disclosure, which may be combined with any other aspect described herein, or portion thereof, extending the filter membrane includes extending a filter membrane sheet sized for providing a plurality of filter membranes between the upper housing plate and the lower housing plate during forming, and separating the filter sets after the heating and sealing of the upper housing plates to the lower housing plates.

In a twentieth aspect of the present disclosure, which may be combined with any other aspect described herein, or portion thereof, the manufacturing method includes separating the plurality of formed upper housing plates and formed lower housing plates, punching at least one aperture for at least one hydrophobic membrane and sealing the at least one hydrophobic membrane across the at least one aperture prior to the heating and sealing of the upper housing plates to the lower housing plates. The punching may occur occurs during the separating.

In a twenty-first aspect of the present disclosure, which may be combined with any other aspect described herein, or portion thereof, the sealing temperature is greater than the softening temperature.

In a twenty-second aspect of the present disclosure, which may be combined with any other aspect described herein, or portion thereof, any of the features, functionality and alternatives described in connection with any one or more of FIGS. 1 to 4 may be combined with any of the features, functionality and alternatives described in connection with any other of FIGS. 1 to 4.

It light of the above aspects and the present disclosure herein, it is an advantage of the present disclosure to provide a filter set that operates with a dual lumen patient line.

It is another advantage of the present disclosure to provide a filter set that filters fresh PD fluid and allows used PD fluid to pass without clogging.

It is a further advantage of the present disclosure to provide a filter set having a filtration capacity that is readily adjustable by varying the size of the filter membrane sheet.

It is yet another advantage of the present disclosure to provide a filter set having a venting function that is readily manufactured and that functions regardless of filter orientation.

It is yet a further advantage of the present disclosure to provide a filter set having a filter housing that may be manufactured using a continuous online deep drawing process.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for peritoneal dialysis system including a patient line filter set having a filter membrane sheet of the present disclosure.

FIG. 2 is a perspective view of one embodiment for a patient line filter set having a filter membrane sheet of the present disclosure.

FIG. 3 is a perspective view of the patient line filter set of FIG. 2 during a patient fill.

FIG. 4 is a perspective view of the patient line filter set of FIG. 2 during a patient drain.

### DETAILED DESCRIPTION

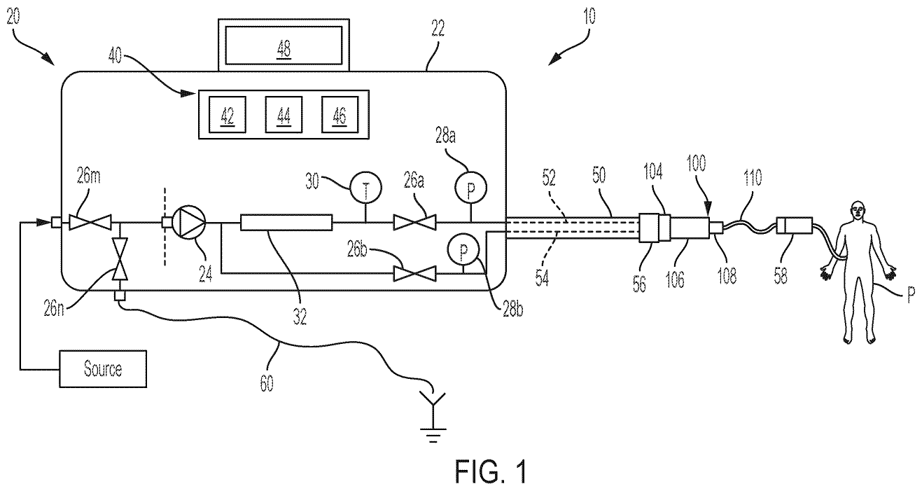

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a filter set 100. If patient line 50 is reusable, the reusable patient line is connected to filter set 100 at the time of treatment. If patient line 50 is instead disposable, filter set 100 is merged into or formed with disposable patient line 50 in one embodiment. In either configuration, a distal end of filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, 26m. 26n, which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b, 26m, 26n include flow-through solenoid valves. Such valves may be two-way or three-way valves. Examples of disposable valves that may be used for valves 26a, 26b, 26m, 26n include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50. Valve 26m is provided to allow selective access to one or more PD fluid source, while valve 26n is provided to allow selective access to a drain, such as a drain container or house drain.

Machine or cycler 20 in the illustrated embodiment also includes pressure sensors, such as pressure sensors 28a, 28b. Pressure sensor 28a is located just downstream from fresh PD fluid valve 26a, while pressure sensor 28b is located just upstream from used PD fluid valve 26b. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from the filter membrane discussed herein during a patient fill. Pressure sensor 28b perhaps more importantly is positioned to sense the pressure of fresh PD fluid downstream from the filter membrane discussed herein during a patient fill.

Pump 24 and valves 26a, 26b in the illustrated embodiment are under the automatic control of control unit 40 provided by machine or cycler 20 of system 10, while pressure sensors 28a, 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater, to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending treatment data to and receiving prescription instructions from a doctor's or clinician's server interfacing with a doctor's or clinician's computer.

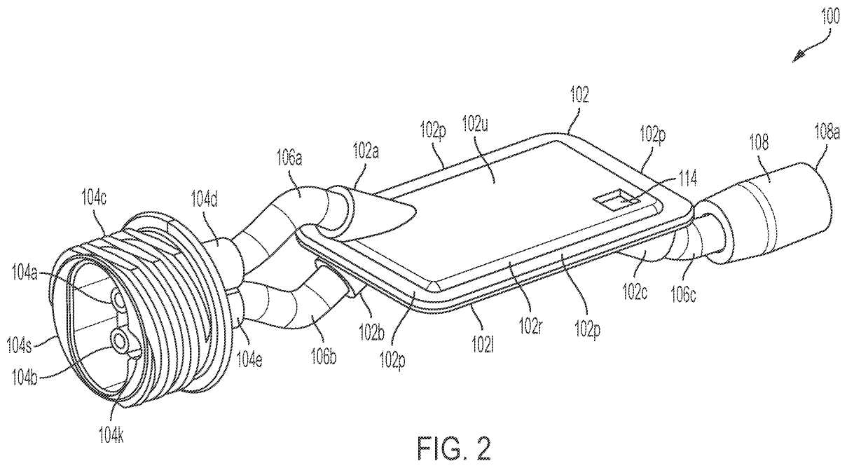

Referring to FIGS. 1 and 2, as mentioned above, fresh and used PD fluid lumens 52 and 54 of dual lumen patient line 50 may again be reusable or disposable. In the instance in which dual lumen patient line 50 is reusable, the lumens terminate with a connector 56 that connects to a lumen-side connector 104 of filter set 100. Lumen-side connector 104 in one embodiment includes a fresh PD fluid port 104a for communication with fresh PD fluid lumen 52 of dual lumen patient line 50 and a used PD fluid port 104b for communicating with used PD fluid lumen 54 of dual lumen patient line 50. Fresh PD fluid port 104a and used PD fluid port 104b are surrounded by a shroud 104s of lumen-side connector 104, wherein shroud 104s is formed with threads 104c for threadingly engaging mating threads of patient line connector 56. The threading of patient line connector 56 to lumen-side connector 104 seals mating ports (not illustrated) of patient line connector 56 to fresh and used PD fluid ports 104a and 104b of the lumen-side connector 104 in one embodiment, e.g., via one or more compressible gasket (not illustrated), such as a silicone or other suitable rubber gasket. In the illustrated embodiment, the front of shroud 104s is formed with a keyed opening 104k. Patient line connector 56 is formed with a mating key so that the patient line connector can only be introduced into shroud 104s in the proper orientation, aligning fresh PD fluid lumen 52 with fresh PD fluid port 104a and used PD fluid lumen 54 with used PD fluid port 104b.

A pair of tubes, including a fresh PD fluid tube 106a and a used PD fluid tube 106b, extend from lumen-side connector 104 to a filter housing 102. Fresh and used PD fluid tubes 106a and 106b are rigid in one embodiment and are bent so as to position lumen-side connector 104 and filter housing 102 relative to each other in a desired manner. In the illustrated embodiment, lumen-side connector 104 is molded to have cylindrical tube receivers 104d, 104e that respectively receive fresh and used PD fluid tubes 106a and 106b. The proximal ends of fresh and used PD fluid tubes 106a and 106b may be sealed respectively within cylindrical tube receivers 104d, 104e ultrasonically, via heat seal and/or adhesively, e.g., via solvent bonding. In an alternative embodiment, fresh and used PD fluid tubes 106a and 106b are molded with lumen-side connector 104.

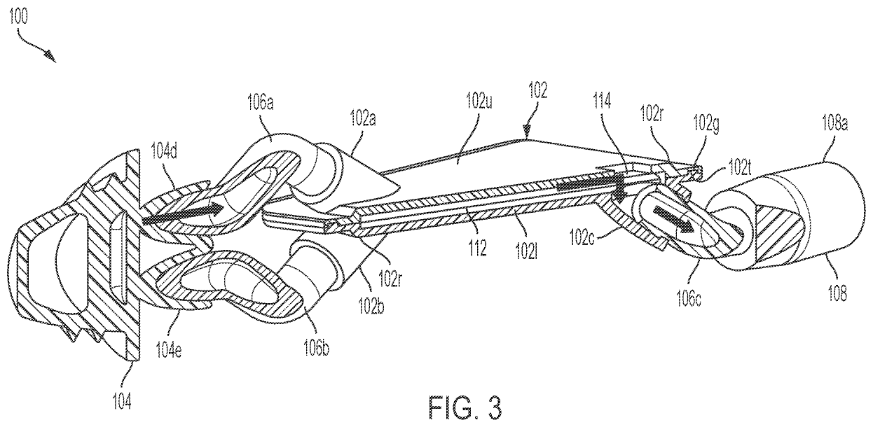

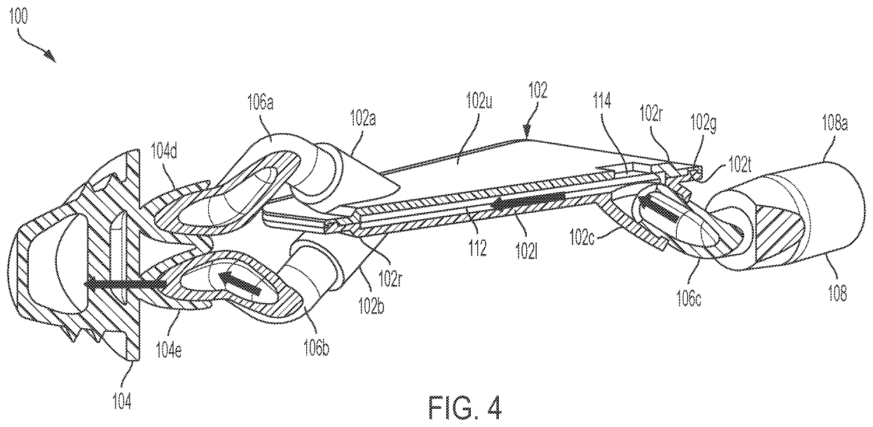

Referring additionally to FIGS. 3 and 4, filter housing 102 is in one embodiment a thin structure having an upper housing plate 102u and a lower housing plate 102l. Each plate 102u, 102l has a slightly raised compartment 102r for receiving PD fluid. Raised compartments 102r are surrounded by perimeters 102p of upper and lower plates 102u, 102l in one embodiment. Plates 102u, 102l may also be molded to each have a respective cylindrical tube receiver 102a, 102b. Cylindrical tube receivers 102a, 102b may be angled to match a bent angle of the fresh or used PD fluid tube 106a, 106b that is received. The distal ends of fresh and used PD fluid tubes 106a and 106b may be sealed respectively within cylindrical tube receivers 102a, 102b ultrasonically, via heat seal and/or adhesively, e.g., via solvent bonding.

Plates 102u, 102l may be molded plastic and a sealed together along their mating perimeters 102p ultrasonically, via heat seal and/or adhesively, e.g., via solvent bonding. The perimeter of one of upper plate 102u or lower plate 102l may be formed with a polygonal, e.g., rectangular, tongue 102t that fits into a like-shaped groove 102g formed in the other upper plate 102u or lower plate 102l. The tongue and groove fitting helps to center upper and lower plates 102u, 102l together during the sealing process.

A filter membrane 112 is sealed along its peripheral edge between perimeters 102p of upper and lower plates 102u, 102l. Filter membrane 112 is a flat sheet in the illustrated embodiment that roughly bisects the raised compartment 102r of upper and lower plates 102u, 102l. Filter membrane 112 may be a sterilizing grade or bacteria reduction hydrophilic membrane, which may be formed with porous walls having a pore size of about 0.2 micron through which the fresh PD fluid flows for further filtration. Filter membrane sheet 112 may be made of, for example, polysulfone or polyethersulfone blended with polyvinylpyrrolidone.

In the illustrated embodiment, lower plate 102l is molded with a second lower cylindrical tube receiver 102c for receiving a transfer set-side tube 106c that extends to a transfer set-side connector 108. The transfer set-side tube 106c, like the fresh and used PD fluid tubes 106a and 106b may be rigid and bent so that transfer set-side connector 108 and filter housing 102 are angled relative to each other in a desired manner. Second lower cylindrical tube receiver 102c may likewise be angled to match an angle formed by transfer set-side tube 106c. Any of filter housing 102, lumen-side connector 104, tubes 106a to 106c, and transfer set-side connector 108 may be made of any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”), or polyurethane (“PU”).

Transfer set-side connector 108 either connects directly to a mating connector of the patient's transfer set 58 or to a mating connector of a short, flexible tube 110 placed between filter housing 102 and the patient's transfer set 58. Transfer set-side connector 108 may include a port (not illustrated) and threaded shroud 108a for a luer type connection to a mating connector. Transfer set-side connector 108 may alternatively simply be a port to which short, flexible tube 110 extends over for welding to the port. Likewise, if dual lumen patient line 50 is disposable, lumen-side connector 104 may alternatively simply include ports, e.g., fresh and used PD fluid ports 104a and 104b, to which fresh and used PD fluid lumens 52 and 54 respectively extend over for welding to the ports.

Filter housing 102 may be starting with an array having blanks for multiple upper plates 102u formed together as a large blank, and blanks for a like-numbered amount of lower plates 102l formed together as a large blank. The array of the large blanks are stacked together and fed through a combined online sealing and deep-drawing process via rollers. At the same time, a large sheet of material for forming multiple filter membranes 112 is fed between the large blank of multiple upper plates 102u and the large blank of multiple lower plates 102l. The deep-drawing process forms upper and lower raised compartments 102r for each filter housing 102. The simultaneous sealing process seals the perimeters 102p of upper and lower plates 102u, 102l for each filter housing 102, thereby sealing filter membranes 112 in place. The large drawn and sealed array is then cut or slit into separate, individual filter housings 102. Cylindrical tube receivers 102a to 102c and the other components of filter set 100 may be secured after separation, e.g., adhesively, to use the above described roller process.

In a simplified embodiment of the manufacturing process for filter housing 102, no deaerating hydrophobic membranes 114 are provided and air is handled as described below. The manufacturing process is then performed as described above, using a single tool in one embodiment for thermoforming upper plates 102u and lower plates 102l and applying a vacuum to each of the large sheets of material. In an embodiment, drawing is performed prior to sealing, because drawing requires lower temperatures. The drawing temperatures used with the tool depend at least in part on the material used for filter housing 102. The temperatures in one embodiment need only be high enough to soften the material for drawing or thermoforming. In certain examples, a tool temperature in the range from 130 to 145° C. may be used for PP, a tool temperature in the range from 120 to 160° C. may be used for PS, a tool a temperature in the range from 70 to 90° C. may be used for PET, while a tool a temperature in the range from 150 to 180° C. may be used for PC. Vacuum pressure applied by the tool for thermoforming in the manufacturing process for filter housing 102 may be selected from a range of, for example, −100 millibar gage (“mbar (g)”) to −900 mbar (g) (−1.5 psig to −13 psig). Sealing using the single tool may be performed subsequent to drawing and require higher temperatures, e.g., 200° C. to 250° C.

For filter housings 102 providing one or more deaerating hydrophobic membrane 114, the manufacturing process may be different. Here, upper plates 102u and lower plates 102l may pass individually through a heating zone and are then thermoformed by applying a tool with a stamp and mold that forms the upper and lower raised compartments 102r. For upper plates 102u, the stamp also cuts out or punches one or more opening for one or more hydrophobic membrane 114, wherein the one or more membrane is then sealed via any technique described herein to the inside of upper plate 102u in one embodiment. Formed upper plates 102u and lower plates 102l and filter membrane sheets 112 are then joined and sealed in a separate sealing tool.

The subsequent finishing steps may be the same regardless of whether or not at least one hydrophobic membrane 114 is provided. Filter housings 102 may be separated from each other using a die cutter. Cuts perpendicular to the belt or roller direction of movement may be made via rollers featuring perpendicular blades. Cylindrical tube receivers 102a to 102c and the other components of filter set 100 may be secured to filter housings 102 after separation, e.g., adhesively. Gluing the hydrophobic membranes 114 to the outsides of filter housings 102 is also an option, but has the disadvantage that the membranes are then not protected from accidental touch and damage by the patient or caregiver.

During a patient fill using filter set 100, fresh PD fluid flows from fresh PD fluid lumen 52, through lumen-side connector 104, through the fresh PD fluid tube 106a and into raised compartment 102r of upper plate 102u. Inside raised compartment 102r, fresh PD fluid is forced under positive pressure from PD fluid pump 24 through filter membrane sheet 112 and into raised compartment 102r of lower plate 102l. Filter membrane sheet 112 is sized to provide sufficient filtration over multiple patient fills while being small enough not to present discomfort to the patient who is likely sleeping during treatment.

The final filtered fresh PD fluid flows from raised compartment 102r of lower plate 102l out second lower cylindrical tube receiver 102c, transfer set-side tube 106c and transfer set-side connector 108 into the patient's transfer set 58, either directly or via short, flexible tube 110. The hydrophilic nature of filter membrane 112 prevents air from migrating across the membrane once the membrane is fully wetted with fresh PD fluid and thus serves a secondary final stage air removal purpose. If needed however, it is contemplated to provide one or more hydrophobic membrane 114 upstream of the filter membrane 112 (from a fresh PD fluid standpoint), e.g., along the surface of upper plate 102u as illustrated in FIGS. 2 to 4. The one or more hydrophobic membrane 114 allows air to be vented to atmosphere prior to the fresh PD fluid flowing through filter membrane sheet 112. Hydrophobic membrane 114 may be constructed for example from polytetrafluoroethylene (“PTFE”).

Used PD fluid removed through the patient's transfer 58 set enters raised compartment 102r of lower plate 102l of filter set 100 via the transfer set-side connector 108 and transfer set-side tube 106c and flows under negative pressure via PD fluid pump 24 from the lower raised compartment 102r, through used PD fluid tube 106b, through used PD fluid port 104b of lumen-side connector 104 and used PD fluid lumen 54, back to PD machine or cycler 20. PD machine or cycler 20 pumps the used PD fluid under positive pressure via PD fluid pump 24 to drain via drain line 60. The used PD fluid does contact the underside of filter membrane 112 sheet but does so in a tangential manner, wherein fibrin, proteins and other particulates within the patient's effluent do not tend to be trapped by or caught on the filter membrane. Filter membrane 112 accordingly remains viable over the course of multiple fills of a treatment prior to being discarded with filter set 100.

It should be understood that various changes and modifications to the presently preferred embodiments described herein will be apparent to those skilled in the art. It is therefore intended that any or all of such changes and modifications may be covered by the appended claims. For example, while a dual lumen patient line 50 is shown operating with fresh and used PD fluid ports 104a and 104b of lumen-side connector 104, the patient line may alternatively be a single lumen patient line, which communicates with a single port within lumen-side connector 104, and wherein that single port is able to communicate with both fresh PD fluid tube 106a and used PD fluid tube 106b. Here, check valves may be sealed and oriented within fresh PD fluid tube 106a and used PD fluid tube 106b, such that fresh PD fluid is prevented from flowing into used PD fluid tube 106b, while used PD fluid is prevented from flowing through fresh PD fluid tube 106a.

1. A peritoneal dialysis (“PD”) system (10) comprising:
a PD machine (20);
a patient line (50) extending from the PD machine (20); and
a filter set (100) including a filter housing (102) having an upper housing plate (102u) and a lower housing plate (102l), and a filter membrane (112) located between the upper housing plate (102u) and the lower housing plate (102l), the filter set (100) further including a lumen-side connector (104) configured to connect to the patient line (50), the lumen-side connector (104) connected to the filter housing (102) via at least one of a fresh PD fluid tube (106a) or a used PD fluid tube (106b).

2. The PD system (10) according to claim 1, wherein the patient line (50) is a dual lumen patient line including a fresh PD fluid lumen (52) placed in fluid communication with the fresh PD fluid tube (106a) of the filter set (100), the dual lumen patient line (50) further including a used PD fluid lumen (54) placed in fluid communication with the used PD fluid tube (106b) of the filter set (100).

3. The PD system (10) according to claim 2, wherein the fresh PD fluid lumen (52) is placed in fluid communication with the fresh PD fluid tube (106a) of the filter set (100) via a fresh PD fluid port (104a) of the lumen-side connector (104), and wherein the used PD fluid lumen (54) is placed in fluid communication with the used PD fluid tube (106b) of the filter set (100) via a used PD fluid port (104b) of the lumen-side connector (104).

4. The PD system (10) according to claim 3, wherein the fresh PD fluid port (104a) and the used PD fluid port (104b) are surrounded by a shroud (104s) of the lumen-side connector (104), the shroud (104s) including threads for mating with a patient line connector.

5. The PD system (10) according to claim 3, wherein the fresh PD fluid port (104a) and the used PD fluid port (104b) are surrounded by a shroud (104s) of the lumen-side connector (104), the shroud (104s) including a keyed opening (104k) for receiving a patient line connector in a desired orientation.

6. The PD system (10) according to claim 3, any of claims 3 to 5, which includes a compressible gasket configured to seal around the fresh and used PD fluid ports (104a, 104b) between the lumen-side connector (104) and a patient line connector.

7. The PD system (10) according to claim 1, wherein at least one of the fresh PD fluid tube (106a) or the used PD fluid tube (106b) is rigid.

8. The PD system (10) according to claim 1, wherein at least one of the fresh PD fluid tube (106a) or the used PD fluid tube (106b) is bent so that the lumen-side connector (104) and the filter housing (102) are positioned relative to each other in a desired manner.

9. The PD system (10) according to claim 1, wherein the filter membrane (112) is a sheet-shaped hydrophilic membrane, and wherein the upper housing plate (102u) and the lower housing plate (102l) include raised compartments (102r) for receiving PD fluid.

10. The PD system (10) according claim 1, wherein the upper housing plate (102u) and the lower housing plate (102l) are formed and sealed along with the filter membrane (112) to form the filter housing (102) via a combined online sealing and deep-drawing process.

11. The PD system (10) according to claim 1, wherein the filter housing (102) is configured such that used PD fluid flows tangentially along the filter membrane (112).

12. The PD system (10) according to claim 1, which includes at least one hydrophobic membrane (114) positioned to vent air from fresh PD fluid upstream from the filter membrane (112).

13. The PD system (10) according to claim 1, wherein the filter set (100) is configured to connect directly to a patient's transfer set, or wherein the filter set (100) includes a flexible tube (110) configured to connect to the patient's transfer set.

14. The PD system (10) according to claim 1, wherein the filter set (100) includes a transfer set-side connector (108) for connecting to a patient's transfer set, the transfer set-side connector (108) connected to the filter housing (102) via a transfer set-side tube (106c).

15. The PD system (10) according to claim 1, wherein the PD machine (20) includes a pressure sensor (28b) positioned to sense pressure of fresh PD fluid downstream from the filter membrane (112) during a patient fill.

16. The PD system (10) according to claim 1, wherein the filter membrane (112) is a sterilizing grade filter membrane or a bacteria reduction filter membrane.

17. A filter set (100) for connecting to a patient line, the filter set (100) comprising:
a filter housing (102) including an upper housing plate (102u) and a lower housing plate (102l);
a filter membrane (112) in the shape of a sheet located between the upper housing plate (102u) and the lower housing plate (102l); and
a lumen-side connector (104) configured to connect to the patient line, the lumen-side connector (104) connected to the filter housing (102) via at least one of a fresh PD fluid tube (106a) or a used PD fluid tube (106b).

18. A method of manufacturing a filter set (100) for connecting to a patient line, the method comprising:
heating at a softening temperature and applying a vacuum to form a plurality of upper housing plates (102u) and lower housing plates (102l);
extending a filter membrane (112) in the shape of a sheet between each of the formed upper housing plates (102u) and the formed lower housing plates (102l); and
heating at a sealing temperature and sealing the upper housing plates (102u) to the lower housing plates (102l) so as to seal in place the filter membranes (112).

19. The manufacturing method of claim 18, wherein extending the filter membrane (112) includes extending a filter membrane sheet sized for providing a plurality of filter membranes (112) between the upper housing plate (102u) and the lower housing plate (102l) during forming, and separating the filter sets (100) after the heating and sealing of the upper housing plates (102u) to the lower housing plates (102l).

20. The manufacturing method of claim 18, which includes separating the plurality of formed upper housing plates (102u) and formed lower housing plates (102l), punching at least one aperture for at least one hydrophobic membrane (114) and sealing at least one hydrophobic membrane (114) across at least one aperture prior to the heating and sealing of the upper housing plates (102u) to the lower housing plates (102l).

21. The manufacturing method of claim 20, wherein the punching occurs during the separating.

22. The manufacturing method of according to claim 18, wherein the sealing temperature is greater than the softening temperature.
